(19) United States
(12) Patent Application Publication
(10) Pub. No.: US 2026/0108160 A1
(43) Pub. Date: Apr. 23, 2026

(54) DEVICE ORIENTATION ALIGNMENT FOR IMPROVED BLOOD PRESSURE ESTIMATION

(71) Applicant: QUALCOMM Incorporated, San Diego, CA (US)

(72) Inventors: Wenhan ZHENG, Amherst, NY (US); Emily Kathryn BROOKS, Amherst, NY (US); John Keith SCHNEIDER, Williamsville, NY (US); Evan Michael BRELOFF, Kenmore, NY (US); Courtney WOLFF, Cheektowaga, NY (US)

(21) Appl. No.: 18/921,263

(22) Filed: Oct. 21, 2024

(51) Int. Cl. A61B 5/021; A61B 5/00

(52) U.S. Cl. CPC A61B 5/021; A61B 5/0095; A61B 5/489; A61B 5/681; A61B 2560/0223

(57) ABSTRACT

Some disclosed methods involve controlling, by a control system, at least one sensor to capture image data corresponding to a current arterial location of an artery based on a current orientation of the apparatus. Some such methods involve obtaining, by the control system, calibration information associated with at least one calibrated arterial location at which the apparatus was previously calibrated, wherein the calibration information includes image data captured by the at least one sensor of the calibrated arterial location. Some such methods involve determining, by the control system, a threshold similarity between the image data corresponding to the current arterial location and the image data corresponding to the calibrated arterial location. Some such methods involve determining, by the control system and based on the threshold similarity, that the current orientation of the apparatus corresponds to the orientation of the apparatus when calibrated at the calibrated arterial location.

### TECHNICAL FIELD

This disclosure relates to device orientation and more specifically to approaches for proper device orientation for cuffless blood pressure measurements.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive, and ambulatory monitoring. Some such devices may measure (or estimate) blood pressure without an inflatable cuff. Although some cuffless devices and systems have previously been deployed, improved devices and systems would be desirable.

### SUMMARY

The systems, methods and devices of this disclosure each have several aspects, no single one of which is solely responsible for the desirable attributes disclosed herein.

One innovative aspect of the subject matter described in this disclosure may be implemented via one or more methods. In some examples, a method may involve controlling, by a control system, at least one sensor to capture image data corresponding to a current arterial location of an artery based on a current orientation of the apparatus. Some such methods involve obtaining, by the control system, calibration information associated with at least one calibrated arterial location at which the apparatus was previously calibrated, wherein the calibration information includes image data captured by the at least one sensor of the calibrated arterial location. Some such methods involve determining, by the control system, a threshold similarity between the image data corresponding to the current arterial location and the image data corresponding to the calibrated arterial location. Some such methods involve determining, by the control system and based on the threshold similarity, that the current orientation of the apparatus corresponds to the orientation of the apparatus when calibrated at the calibrated arterial location.

Other innovative aspects of the subject matter described in this disclosure may be implemented in an apparatus. The apparatus may include a user interface system that includes a display, a fingerprint sensor system, a memory system and a control system configured for communication with the display, the fingerprint sensor system and the memory system. The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. In some implementations, a mobile device (such as a wearable device, a cellular telephone, etc.) may be, or may include, at least part of the apparatus.

According to some examples, the control system may be configured to control at least one sensor of an apparatus to capture image data corresponding to a current arterial location of an artery based on a current orientation of the apparatus. The control system may be configured to obtain calibration information associated with at least one calibrated arterial location at which the apparatus was previously calibrated, wherein the calibration information includes image data captured by the at least one sensor of the calibrated arterial location. The control system may be configured to determine a threshold similarity between the image data corresponding to the current arterial location and the image data corresponding to the calibrated arterial location. The control system may be configured to determine, based on the threshold similarity, that the current orientation of the apparatus corresponds to the orientation of the apparatus when calibrated at the calibrated arterial location.

Some or all of the operations, functions and/or methods described herein may be performed by one or more devices according to instructions (e.g., software) stored on one or more non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon.

For example, the software may include instructions for controlling one or more devices to perform one or more methods. Some such methods may involve controlling, by a control system, at least one sensor to capture image data corresponding to a current arterial location of an artery based on a current orientation of the apparatus. Some such methods involve obtaining, by the control system, calibration information associated with at least one calibrated arterial location at which the apparatus was previously calibrated, wherein the calibration information includes image data captured by the at least one sensor of the calibrated arterial location. Some such methods involve determining, by the control system, a threshold similarity between the image data corresponding to the current arterial location and the image data corresponding to the calibrated arterial location. Some such methods involve determining, by the control system and based on the threshold similarity, that the current orientation of the apparatus corresponds to the orientation of the apparatus when calibrated at the calibrated arterial location.

### BRIEF DESCRIPTION OF THE DRAWINGS

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

FIG. 1 shows an example of a blood pressure (BP) monitoring device according to some disclosed implementations.

FIGS. 2A-2C are example diagrams showing example variations in the measurement of arterial cross-sectional area that may result from changes in device orientation.

FIG. 3 is a block diagram that shows example components of an apparatus according to some disclosed implementations.

FIG. 4A shows an example blood pressure (BP) monitoring device as one implementation of the apparatus.

FIG. 4B is a flow diagram that shows examples of some disclosed operations relating to single-point device calibration.

FIG. 4C is a flow diagram that shows examples of some disclosed operations relating to single-point device alignment.

FIG. 5A shows an example blood pressure (BP) monitoring device as one implementation of the apparatus.

FIG. 5B is a flow diagram that shows examples of some disclosed operations relating to multi-point device calibration.

FIG. 5C is a flow diagram that shows examples of some disclosed operations relating to multi-point device alignment.

FIG. 6 is a flow diagram that shows examples of some disclosed operations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Non-invasive health monitoring devices, such as ultrasonic-based devices and photoacoustic plethysmography (PAPG)-capable devices, have various potential advantages over more invasive health monitoring devices, such as cuff-based or catheter-based blood pressure (BP) measurement devices. Such non-invasive health monitoring devices may offer the convenience of being wearable and able to perform cuffless BP estimations on demand and in real-time. A wearable device may initially be calibrated for BP estimation based on a known device orientation relative to an area of interest (e.g., an arterial location). After calibration, the wearable device may be used to determine future BP estimations at various times, as needed and on demand. When such BP estimations are made, having the wearable device be in an orientation that is the same (or substantially similar) to the orientation of the wearable device at the time of calibration can be especially useful for improving the accuracy of the BP estimations. However, by their very nature, wearable devices are prone to frequent changes in device orientation due to daily activity. Such changes to orientation may dramatically affect the accuracy with which arterial properties, such as arterial cross-section area, are measured. As a result, the accuracy of BP estimations made based on such measured arterial properties may also suffer.

Some disclosed devices are configured to determine when a device orientation of the device is or is not aligned with a device orientation of the device at the time of calibration, and perform operations accordingly. If a determination is made that the device orientation is aligned, the operations may include estimating a blood pressure. Alternatively, if a determination is made that the device orientation is not aligned, the operations may include the provision of user instructions to re-align the orientation of the device or algorithmically compensating a blood pressure estimation based on the misalignment.

According to some implementations, the device may capture and store image data (“calibration image data”) of an artery of interest at the time of device calibration. For example, if the device is a bracelet, then the device may image a portion of the radial artery along a wrist on which the bracelet is worn. When future BP estimations are requested, the device may be configured to use the stored image data to determine if its current device orientation is aligned with its device orientation at the time of calibration. For example, to evaluate alignment, the device may capture image data (“test image data”) of the artery of interest based on its current device orientation. The device may compare the calibration image data and the test image data to determine whether and to what degree its orientation is aligned. Suppose the device is determined to be aligned. In that case, the device may be configured to measure arterial properties, such as arterial cross-sectional area, which may then be used for BP estimation. Alternatively, if the device is determined to be misaligned, then the device may perform operations to align the device's orientation. According to some implementations, a display associated with the device may provide user instructions to aid in aligning the device orientation.

Particular implementations of the subject matter described in this disclosure can be implemented to realize one or more of the following potential advantages. Some disclosed devices are able to facilitate proper device orientation to help improve the accuracy of blood pressure (BP) properties, such as arterial cross-sectional area. As a result, BP estimations, which may be made based on such arterial properties, may also improve. This awareness of device orientation allows the device to maintain consistent sensor positioning relative to arterial locations of interest, which is crucial for reliable measurements over time. The device may also prompt a user to adjust the device to correct the device's orientation, thereby reducing BP estimation errors caused by improper wear. Additionally, the device may be able to compensate for minor misalignments automatically. This capability may particularly be valuable during continuous monitoring, as it can account for natural shifts in position during daily activities or sleep. By ensuring optimal sensor orientation, the device can provide more accurate measurements of arterial properties, such as arterial cross-sectional area, which are useful for improving the accuracy of cuffless blood pressure estimation techniques.

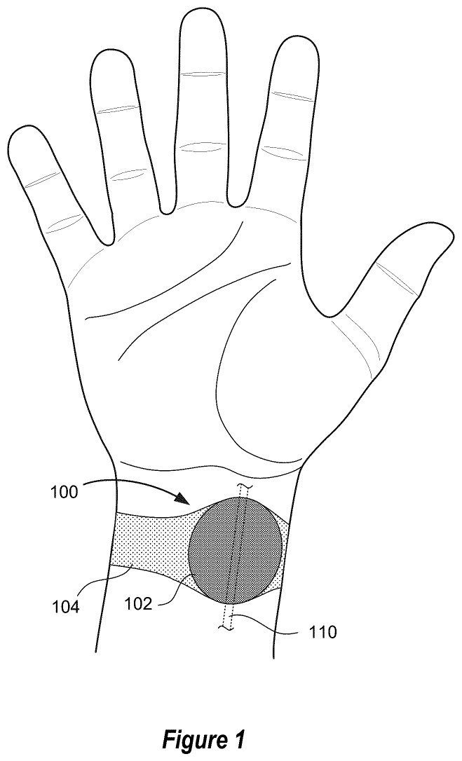

FIG. 1 shows an example device (e.g., an ambulatory monitoring device) 100 designed to be worn around a wrist, according to some implementations. In the illustrated example, the device 100 includes a housing 102 integrally formed with, coupled with, or otherwise integrated with, a band (or strap) 104. In this example, the device 100 is coupled around the wrist such that one or more sensors (e.g., photoacoustic, ultrasonic, optical, camera, etc.) within the housing 102 will measure arterial properties and make BP estimations along a stretch of an artery 110.

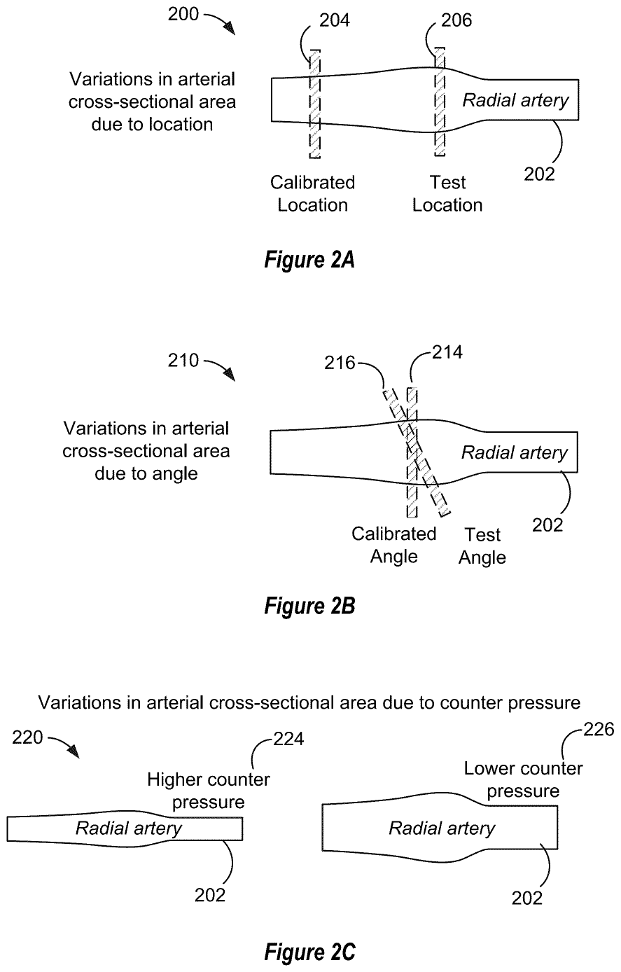

As mentioned, deviations in the orientation of the device 100 from the orientation of the device at the time of calibration may hinder accurate measurements of arterial properties, such as arterial cross-sectional area. According to some implementations, an orientation of the device may be defined based on a set of parameters, including device location, angle, and counter pressure relative to a surface, such as a human wrist. For example, FIG. 2A illustrates an example diagram 200 showing measurement variations in a cross-sectional area of an artery 202 due to a change in device location relative to a wrist. In the example of FIG. 2A, the device 100 may have been calibrated such that one or more sensors of the device 100 may correspond to an arterial location 204 along a stretch of the artery 202. In this example, any deviation in the orientation of the device 100 may result in the sensors of the device 100 corresponding to a different location, such as a location 206 along the artery 202. As a result, measurement of a cross-sectional area of the artery 202 at the different location 206 may vary from the measurement of the cross-sectional area of the artery 202 at the location 204, for example, due to arterial morphology, despite there being no change in arterial blood pressure.

In another example, FIG. 2B illustrates an example diagram 210 showing measurement variations in a cross-sectional area of the artery 202 due to a change in device angle. In the example of FIG. 2B, the device 100 may have been calibrated such that one or more sensors of the device 100 may correspond to an angle 214 with respect to the artery 202. In this example, any deviation in the orientation of the device 100 may result in the sensors of the device 100 corresponding to a different angle, such as an angle 216 with respect to the artery 202. As a result, measurement of a cross-sectional area of the artery 202 based on the different angle 216 may vary from the measurement of the cross-sectional area of the artery 202 based on the angle 214, for example, due to arterial morphology, despite there being no change in arterial blood pressure.

In yet another example, FIG. 2C illustrates an example diagram 220 showing measurement variations in a cross-sectional area of an artery 202 due to device counter pressure. For example, a tightness associated with the band 104 of the device 100 may play a crucial role in exerting counter pressure on the underlying tissue and artery 202, which can significantly impact the measurement of arterial cross-sectional area. The counter pressure applied by the band 104 can directly affect how sensors interact with the arterial wall and surrounding tissues. As shown in the example of FIG. 2C, if the band 104 is too tight, the higher counter pressure 224 can increase tissue compression and reduce the cross-sectional area of the artery 202. In contrast, if the band 104 is too loose, the lower counter pressure 226 can reduce tissue compression and increase the cross-sectional area of the artery 202. Accordingly, maintaining consistent counter pressure between calibration and test can be important for reliably measuring arterial properties.

In general, variability in arterial cross-sectional area due to changes in device orientation (e.g., location, angle, counter pressure relative to a wrist) can make it challenging to determine a reliable relationship between arterial cross-sectional area and blood pressure. Without an accurate relationship between arterial cross-sectional area and blood pressure, the accuracy of test BP estimations may be compromised. Accordingly, it is desirable to ensure the device 100 measures arterial properties at some area of interest along an artery based on a device orientation that remains consistent between calibration and test. Such consistency helps ensure that measurements, such as arterial cross-sectional area, are reliable and may be applied to more accurately estimate BP.

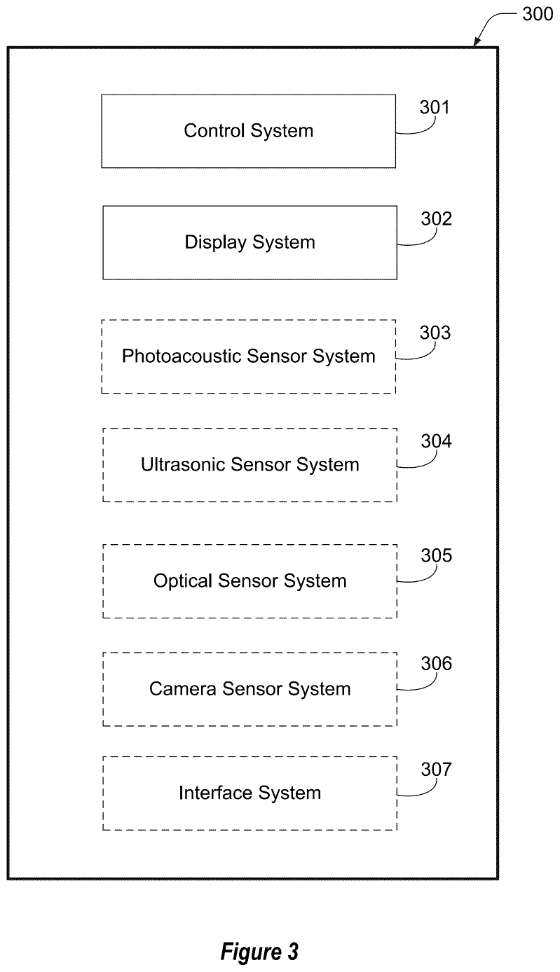

FIG. 3 is a block diagram that shows example components of an apparatus 300 according to some disclosed implementations. The apparatus 300 may be used in a variety of different contexts, some examples of which are disclosed herein. For example, in some implementations, a mobile device may include at least a portion of the apparatus 300. In some implementations, a wearable device (e.g., the device 100) may include at least a portion of the apparatus 300. The wearable device may, for example, be a smart watch, a bracelet, an armband, a wristband, a ring, a headband, or a patch. Accordingly, in some examples the apparatus 300 may be configured to be worn by, or attached to, a person. In some implementations, the control system 301 may reside in more than one device. For example, a portion of the control system 301 may reside in a wearable device and another portion of the control system 301 may reside in another device, such as a mobile device (e.g., a smartphone).

In this example, the apparatus 300 includes at least a control system 301, a display system 302, and at least one of a photoacoustic sensor system 303, an ultrasonic sensor system 304, an optical sensor system 305, or a camera sensor system 306. According to some examples, the apparatus 300 may be configured to obtain measurements (e.g., arterial properties) that can be used for BP estimation. Some implementations of the apparatus 300 may include an interface system 307. As with other disclosed implementations, in some alternative implementations, the apparatus 300 may include more components, fewer components, or different components.

The control system 301 may include one or more general purpose single- or multi-chip processors, digital signal processors (DSPs), application-specific integrated circuits (ASICs), field programmable gate arrays (FPGAs), or other programmable logic devices, discrete gates or transistor logic, discrete hardware components, or combinations thereof. The control system 301 may also include (and/or be configured for communication with) one or more memory devices, such as one or more random access memory (RAM) devices, read-only memory (ROM) devices, etc. Accordingly, the apparatus 300 may have a memory system that includes one or more memory devices, though the memory system is not shown in FIG. 1.

The control system 301 may be configured to control the display system 302, the photoacoustic sensor system 303, the ultrasonic sensor system 304, the optical sensor system 305, and the camera sensor system 306, as described herein. For example, the control system 301 may be configured to receive and process data from the photoacoustic sensor system 303, the ultrasonic sensor system 304, the optical sensor system 305, and the camera sensor system 306. In some implementations, functionality of the control system 301 may be partitioned between one or more controllers or processors, such as a dedicated sensor controller and an applications processor of a mobile device.

According to some implementations, the control system 301 may be configured to control the photoacoustic sensor system 303. The photoacoustic sensor system 303 may include components that work together to detect and measure acoustic signals generated by the photoacoustic effect. Such components can include an interface, a light source system, and a receiver system. For example, the light source system may include a laser or light-emitting diode (LED) that emits pulsed or modulated light at specific wavelengths. When the light source system emits pulses or modulated light onto a sample (e.g., arterial location), some of the light may be absorbed by the sample. This absorbed light energy may be converted into heat, causing rapid thermal expansion and contraction of the sample and surrounding gas. These thermal fluctuations may generate sound waves that propagate through the sample. The receiver system (e.g., an acoustic detector) may receive these sound waves. The detected signals may then be processed and analyzed to extract relevant information from background noise. By varying the wavelength of the light source system and measuring the corresponding acoustic signals, a spectrum of the sample's absorption characteristics can be obtained, providing information about its composition and properties. According to some examples, the control system 301 may be configured to detect an artery within the sample and determine its arterial properties (e.g., arterial cross-sectional area) based on the detected signals. In some examples, the control system 301 may be configured to estimate blood pressure within the artery based on the arterial properties.

In some implementations, the control system 301 may be configured to control the photoacoustic sensor system 303 to perform arterial tomographic mapping by leveraging the photoacoustic effect to generate high-resolution, three-dimensional (3D) images of blood vessels. For example, the photoacoustic sensor system 303 may direct short pulses of laser light directed at tissue containing an arterial location of interest. The hemoglobin in the blood can absorb this light energy, causing rapid thermal expansion and contraction, which can generate broadband ultrasound waves. The receiver system may then detect these waves (e.g., an acoustic detector). The photoacoustic sensor system 303 can capture the spatial and temporal distribution of these acoustic waves. In some implementations, the data received by the receiver system may be processed using reconstruction algorithms to create detailed 3D images of the arterial structure and corresponding properties, allowing for high-resolution imaging of arteries and microvasculature.

According to some implementations, the control system 301 may be configured to control the ultrasonic sensor system 304. The ultrasonic sensor system 304 may include components, such as an interface, a source system, and a receiver system, that work together to transmit ultrasonic waves to a sample (e.g., arterial location). The receiver system may receive ultrasonic waves reflected back from the sample. According to some examples, the control system 301 may be configured to detect an artery within the sample and determine its arterial properties (e.g., arterial cross-sectional area) based on the detected signals. In some examples, the control system 301 may be configured to estimate blood pressure within the artery based on the arterial properties.

In some implementations, the ultrasonic sensor system 304 may include a component (e.g., a piezoelectric transducer) that is capable of both transmitting and receiving ultrasonic waves. In such implementations, the control system 301 may be configured to control the ultrasonic sensor system 304 to perform pulse-echo ultrasound techniques that use high-frequency sound waves to image internal vascular structures and measure distances. For example, the ultrasonic sensor system 304 may generate a short burst of ultrasound waves. These waves can travel through a sample (e.g., artery) being examined until they encounter a segmentation between materials with different acoustic properties, such as the boundary between soft tissue and bone. At these segmentations, part of the sound wave is reflected back and detected by the ultrasonic sensor system 304 as an echo. By analyzing the timing, amplitude, and frequency of these echoes, the ultrasonic sensor system 304 can create detailed images of internal vascular structures and provide accurate measurements of thickness and distance.

According to some implementations, the control system 301 may be configured to control the optical sensor system 305. The optical sensor system 305 may include components, such as an interface, a light source system, and a receiver system, that work together to emit light to a sample (e.g., arterial location). The receiver system may receive light reflected back from the sample. According to some examples, the control system 301 may be configured to detect an artery within the sample and determine its arterial properties (e.g., arterial cross-sectional area) based on the detected signals. In some examples, the control system 301 may be configured to estimate blood pressure within the artery based on the arterial properties. In general, the optical sensor system 305 may apply various approaches for blood pressure estimation, including photoplethysmography (PPG), pulse transit time (PTT), and pulse wave analysis, to name some examples. In some implementations, the control system 301 may be configured to control the optical sensor system 305 to create detailed images of internal vascular structures and provide accurate measurements of thickness and distance. In some implementations, the control system 301 may be configured to control the optical sensor system 305 to detect dermatoglyphic skin patterns, such as skin texture found on the inside of a wrist.

According to some implementations, the control system 301 may be configured to control the camera sensor system 306. The camera sensor system 306 may allow the control system 301 to image skin features, such as dermatoglyphic skin patterns, such as skin texture found on the inside of a wrist.

Some implementations of the apparatus 300 may include the interface system 307. In some examples, the interface system 307 may include a wireless interface system. In some implementations, the interface system 307 may include a user interface system, one or more network interfaces, one or more interfaces between the control system 301 and a memory system and/or one or more interfaces between the control system 301 and one or more external device interfaces (e.g., ports or applications processors), or combinations thereof. According to some examples in which the interface system 307 is present and includes a user interface system, the user interface system may include a microphone system, a loudspeaker system, a haptic feedback system, a voice command system, one or more displays, or combinations thereof. According to some examples, the interface system 307 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

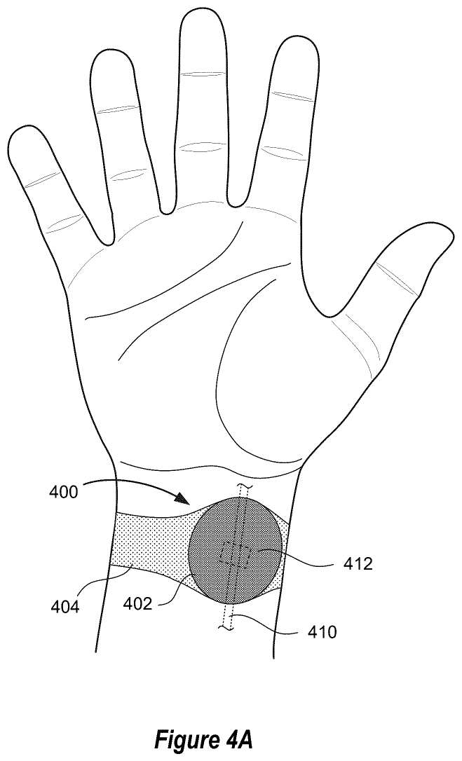

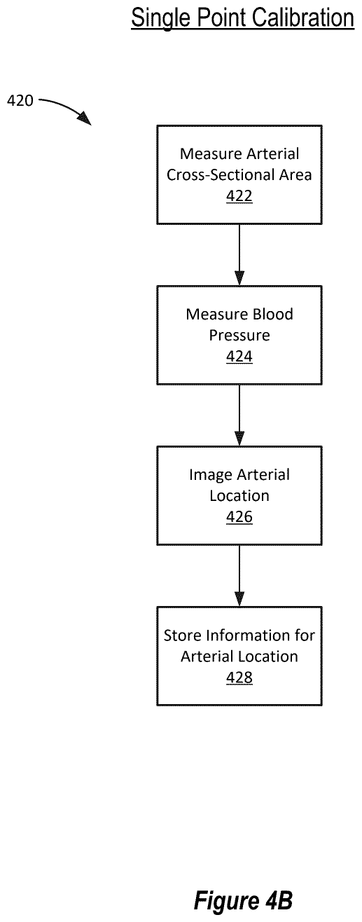

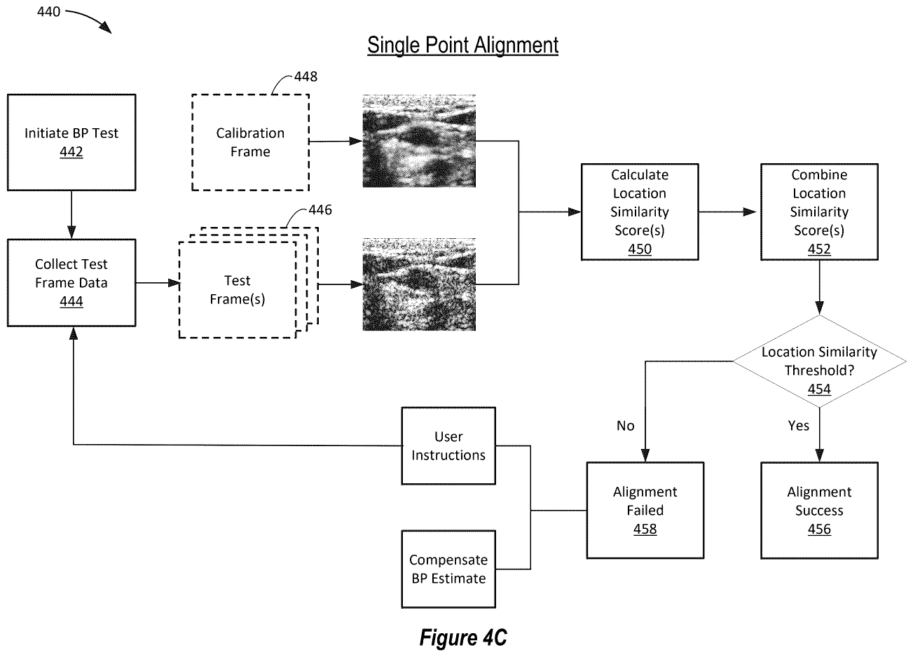

FIG. 4A shows an example blood pressure (BP) monitoring device 400 as one implementation of the apparatus 300. The BP monitoring device 400 may be designed to be worn around a wrist, according to some implementations. In the illustrated example, the BP monitoring device 400 includes a housing 402 integrally formed with, coupled with, or otherwise integrated with, a band (or strap) 404. In this example, the BP monitoring device 400 is coupled around the wrist such that one or more sensors (e.g., photoacoustic, ultrasonic, optical, camera, etc.) within the housing 402 will measure arterial properties and make BP estimations at arterial locations that corresponds to some portion of an artery 410. In this example, the BP monitoring device 400 may be calibrated for a single arterial location 412, as described in reference to FIG. 4B.

FIG. 4B is a flow diagram that shows examples of some disclosed operations relating to single-point device calibration. The blocks of FIG. 4B may, for example, be performed (at least in part) by the control system 301 of FIG. 3. As with other methods disclosed herein, the method 420 outlined in FIG. 4B may include more or fewer blocks than indicated. Moreover, the blocks of methods disclosed herein are not necessarily performed in the order indicated. In some examples, some blocks of methods disclosed herein may be performed concurrently or substantially concurrently.

In this example, the method 420 begins with block 422. Block 422 may, for example, involve calibrating the apparatus 300, implemented as the BP monitoring device 400, at the arterial location 412. For example, the calibration may be performed when the BP monitoring device 400 is first used and at periodic time intervals to maintain accuracy. In some implementations, when calibrating the BP monitoring device 400, one or more sensors of the BP monitoring device 400 may be used to determine (or measure) arterial cross-sectional area for the arterial location 412. For example, the arterial cross-sectional area may be determined at both systole and diastole. Depending on the sensor implementation, the arterial cross-sectional area may be determined based on generally known techniques, such as photoacoustic imaging, ultrasound-based methods, optical methods, etc.

In this example, block 424 involves determining a ground truth blood pressure for the arterial location 412. According to some implementations, the ground truth blood pressure for the arterial location 412 may be determined using a traditional BP monitoring device, such as a cuff-based BP monitoring device.

In this example, block 426 involves imaging the arterial location 412. The arterial location 412 may be imaged using any of the techniques described herein, such as arterial tomographic mapping based on the photoacoustic sensor system 303, imaging internal microvascular structures based on the ultrasonic sensor system 304, or dermatoglyphic skin patterns based on the optical sensor system 305 and/or the camera sensor system 306, to name some examples. According to some implementations, when imaging the arterial location 412, one or more sensors of the BP monitoring device 400 may capture image data (or image segment) of the arterial location 412. The image segment may correspond to a set of frames representing the arterial location 412 that were captured by sensors of the BP monitoring device 400 over some period of time (e.g., 5 seconds, 10 seconds, etc.). In some implementations, the set of frames of the arterial location 412 may be combined into a single calibration frame of the arterial location 412. In some implementations, the set of frames may be combined based on an image averaging technique, which can help reduce noise and improve accuracy. The technique may involve stacking the set of frames and calculating average pixel values across the frames to produce a single, less noisy calibration frame that corresponds to the arterial location 412.

In this example, block 428 involves storing calibration information in association with the arterial location 412, including various arterial measurements, such as the arterial cross-sectional area measured in block 422, the ground truth blood pressure measured in block 424, and the calibration frame determined for the arterial location 412 in block 426. Such calibration information may be used for aligning the BP monitoring device 400 when performing future BP estimations, as described in reference to FIG. 4C.

FIG. 4C is a flow diagram that shows examples of some disclosed operations relating to single-point device alignment. The blocks of FIG. 4C may, for example, be performed (at least in part) by the control system 301 of FIG. 3, implemented as the BP monitoring device 400. As with other methods disclosed herein, the method 440 outlined in FIG. 4C may include more or fewer blocks than indicated. Moreover, the blocks of methods disclosed herein are not necessarily performed in the order indicated. In some examples, some blocks of methods disclosed herein may be performed concurrently or substantially concurrently.

In this example, the method 440 begins with block 442. Block 442 may involve initiating a blood pressure (BP) test. For example, the BP test may be initiated in response to the BP monitoring device 400 being worn or due to an instruction to initiate the BP test while a user is wearing the BP monitoring device 400.

In this example, block 444 may involve imaging a current arterial location. For example, the current arterial location may be imaged using one or more sensors of the BP monitoring device 400 (e.g., photoacoustic, ultrasonic, optical, camera, etc.). The current arterial location may correspond to some portion of an artery as determined based on a current orientation of the BP monitoring device 400. In general, the current arterial location may be imaged using any of the techniques described herein, such as those involving arterial tomographic mapping, internal microvascular structures, or dermatoglyphic skin patterns, to name some examples. According to some implementations, when imaging the current arterial location, one or more sensors of the BP monitoring device 400 may capture at least one image segment of the current arterial location. The image segment may correspond to a set of frames 446 of the current arterial location that were captured by sensors of the BP monitoring device 400 over some period of time (e.g., 5 seconds, 10 seconds, etc.).

In this example, block 450 involves determining a location similarity score for each of the set of frames 446 for the current arterial location with respect to the calibration frame 448 that was determined for the arterial location 412 at block 426 of FIG. 4B. The location similarity score for each frame 446 provides a numerical measure of a level of similarity between that frame and the calibration frame 448. In some implementations, a location similarity score may be determined based on a Structural Similarity Index Measure (SSIM) technique. For example, the control system 301 may apply the SSIM technique to measure image similarity by comparing local patterns of pixel intensities across luminance, contrast, and structure. While the SSIM technique is provided as one example approach for measuring similarity between a pair of frames, any generally known approach for evaluating a pair of frames to measure similarity, such as Mean Squared Error (MSE), histogram comparison techniques, and deep learning-based methods, to name some examples.

In this example, block 452 involves combining the location similarity scores for the set of frames 446. According to some implementations, the location similarity scores are averaged to produce a combined location similarity score.

In this example, block 454 involves determining whether the combined location similarity score satisfies a threshold value. For example, an SSIM score may range from −1 to 1, where 1 indicates perfect similarity between a pair of frames. For example, the threshold value could be 0.5, 0.75, or 0.9. In this example, if it is determined in block 454 that the combined location similarity score satisfies the threshold value, method 440 may proceed to block 456. Alternatively, if it is determined in block 454 that the combined location similarity score does not satisfy the threshold value, method 440 may proceed to block 458.

In this example, block 456 may involve confirming that a current orientation of the BP monitoring device 400 is aligned with an orientation of the BP monitoring device 400 at the time of calibration. In this example, the satisfaction of the location similarity threshold at block 454 indicates that the current arterial location imaged by the BP monitoring device 400 at the time of initiating the BP test in block 442 corresponds to the arterial location 412 that was imaged by the BP monitoring device 400 at the time of BP calibration. Based on the correspondence, a determination can be made that the current orientation of the BP monitoring device 400 is aligned with the orientation of the BP monitoring device 400 at the time of calibration. According to some implementations, upon confirming alignment, the BP monitoring device 400 may be configured to perform a blood pressure (BP) estimation. In some implementations, the BP estimation may be determined based on the ground truth BP associated with the arterial location 412 and a measurement of an arterial cross-sectional area associated with the current arterial location. For example, the BP estimation may be determined based, in part, by mapping a percentage change in the arterial cross-sectional area for the current arterial location to a percentage change in BP for the arterial location 412.

In this example, block 458 may involve confirming that a current orientation of the BP monitoring device 400 is not aligned with an orientation of the BP monitoring device 400 at the time of calibration. According to some embodiments, user instructions for re-adjusting the BP monitoring device 400 may be provided. For example, the control system 301 may provide such instructions via the display system 302 associated with the BP monitoring device 400. In some implementations, the user instructions may include text-, voice-, or haptic-based live feedback that instructs the user to make changes to the orientation of the BP monitoring device 400. The instructions may ask the user to adjust the location or angle of the BP monitoring device 400. As another example, the instructions may ask the user to increase or decrease the tightness of a band associated with the BP monitoring device 400 to adjust counter pressure. As the user re-positions the BP monitoring device 400 to correspond to a different arterial location, method 440 may proceed again to block 444, which may involve imaging and evaluating the different arterial location for alignment, as described above. According to some implementations, user instructions asking the user to adjust the orientation of the BP monitoring device 400 may continue to be provided until a determination is made that the orientation of the BP monitoring device 400 is aligned.

According to some embodiments, rather than asking the user to re-position the BP monitoring device 400, the control system 301 may compensate (or adjust) the current BP estimate based on a relationship between the current orientation of the BP monitoring device 400 and a calibrated arterial location that most closely aligns with the current orientation.

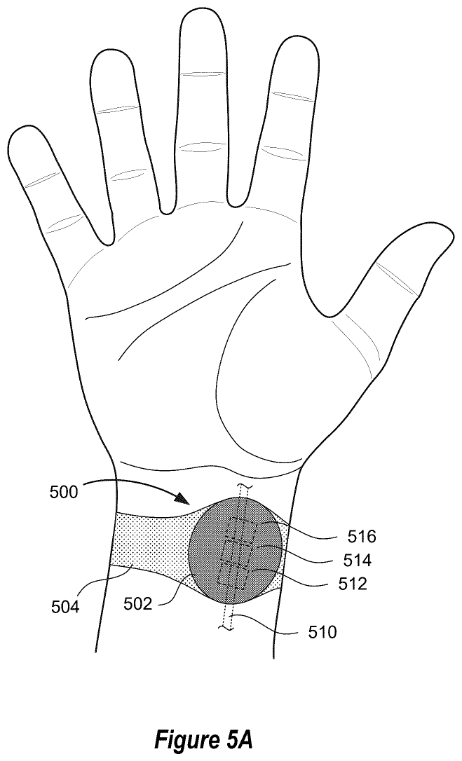

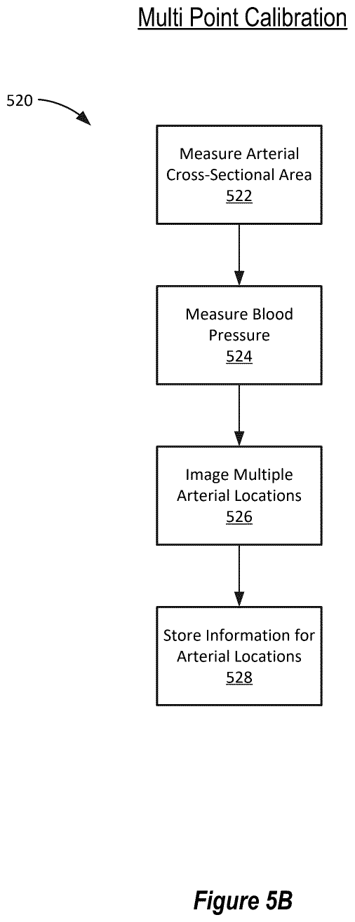

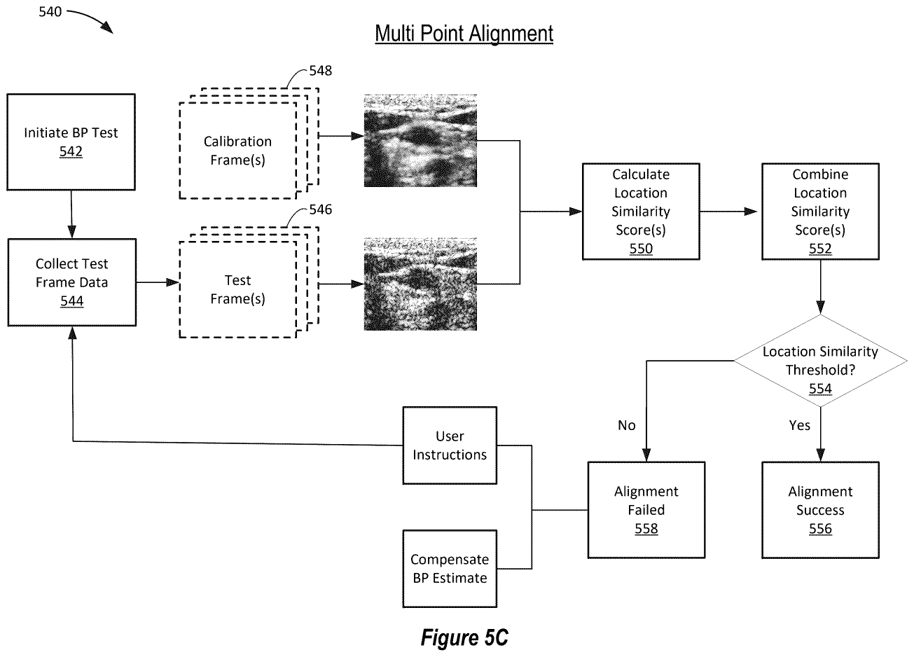

FIG. 5A shows an example blood pressure (BP) monitoring device 500 as one implementation of the apparatus 300. The BP monitoring device 500 may be designed to be worn around a wrist, according to some implementations. In the illustrated example, the BP monitoring device 500 includes a housing 502 integrally formed with, coupled with, or otherwise integrated with, a band (or strap) 504. In this example, the BP monitoring device 500 is coupled around the wrist such that one or more sensors (e.g., photoacoustic, ultrasonic, optical, camera, etc.) within the housing 502 will measure arterial properties and make BP estimations at arterial locations that each correspond to some portion of an artery 510. In this example, the BP monitoring device 500 is calibrated at multiple arterial locations 512, 514, and 516, as described in reference to FIG. 5B. Depending on the implementation, the number of arterial locations at which the BP monitoring device 500 is calibrated may vary. For example, in some implementations, the BP monitoring device 500 may be calibrated at two arterial locations. In some implementations, the BP monitoring device 500 may be calibrated at multiple arterial locations (e.g., 5, 7, 10, etc.) across and along a wrist on which the BP monitoring device 500 is worn.

FIG. 5B is a flow diagram that shows examples of some disclosed operations relating to multi-point device calibration. The blocks of FIG. 5B may, for example, be performed (at least in part) by the control system 301 of FIG. 3. As with other methods disclosed herein, the method 520 outlined in FIG. 5B may include more or fewer blocks than indicated. Moreover, the blocks of methods disclosed herein are not necessarily performed in the order indicated. In some examples, some blocks of methods disclosed herein may be performed concurrently or substantially concurrently.

In this example, the method 520 begins with block 522. Block 522 may, for example, involve calibrating the apparatus 300, implemented as the BP monitoring device 500, at each of the arterial locations 512, 514, and 516. For example, the calibration may be performed when the BP monitoring device 500 is first used and at periodic time intervals to maintain accuracy. In some implementations, when calibrating the BP monitoring device 500, one or more sensors of the BP monitoring device 500 may be used to determine (or measure) a respective arterial cross-sectional area for each arterial location 512, 514, and 516. For example, the arterial cross-sectional area may be determined at both systole and diastole. Depending on the sensor implementation, the arterial cross-sectional area may be determined based on generally known techniques, such as photoacoustic imaging, ultrasound-based methods, optical methods, etc.

In this example, block 524 involves determining a ground truth blood pressure corresponding to the arterial locations 512, 514, and 516. According to some implementations, the ground truth blood pressure for all of the arterial locations may be determined using a traditional BP monitoring device, such as a cuff-based BP monitoring device. According to some implementations, the same ground truth blood pressure is used for all of the arterial locations 512, 514, and 516.

In this example, block 526 involves imaging each of the multiple arterial locations 512, 514, and 516. Each of the arterial locations may be imaged using any of the techniques described herein, such as arterial tomographic mapping based on the photoacoustic sensor system 303, imaging internal microvascular structures based on the ultrasonic sensor system 304, or dermatoglyphic skin patterns based on the optical sensor system 305 and/or the camera sensor system 306, to name some examples. According to some implementations, when imaging an arterial location, one or more sensors of the BP monitoring device 500 may capture image data (or image segment) of the arterial location. The image segment may correspond to a set of frames representing the arterial location that were captured by sensors of the BP monitoring device 500 over some period of time (e.g., 5 seconds, 10 seconds, etc.). In some implementations, the set of frames corresponding to the arterial location may be combined into a single calibration frame representing the arterial location. Thus, a corresponding image segment may be determined for each arterial location 512, 514, and 516. In some implementations, the set of frames associated with an image segment may be combined, for example, based on an image averaging technique, as described above. Accordingly, for each arterial location 512, 514, and 516, a single corresponding calibration frame may be determined based on the averaging.

In this example, block 528 involves storing calibration information in association with each of the arterial locations 512, 514, and 516. For example, for each arterial location 512, 514, and 516, the stored calibration information can include various arterial measurements, such as the corresponding arterial cross-sectional area measured for that arterial location in block 522, the ground truth blood pressure measured in block 524, and the corresponding calibration frame determined for the arterial location in block 526. Such calibration information may be used for aligning the BP monitoring device 500 based on any one of the arterial locations 512, 514, or 516 when performing future BP estimations, as described in reference to FIG. 5C.

FIG. 5C is a flow diagram that shows examples of some disclosed operations relating to multi-point device alignment. The blocks of FIG. 5C may, for example, be performed (at least in part) by the control system 301 of FIG. 3. As with other methods disclosed herein, the method 540 outlined in FIG. 5C may include more or fewer blocks than indicated. Moreover, the blocks of methods disclosed herein are not necessarily performed in the order indicated. In some examples, some blocks of methods disclosed herein may be performed concurrently or substantially concurrently.

In this example, the method 540 begins with block 542. Block 542 may involve initiating a blood pressure (BP) test. For example, the BP test may be initiated in response to the BP monitoring device 500 being worn or due to an instruction to initiate the BP test while a user is wearing the BP monitoring device 500.

In this example, block 544 may involve imaging a current arterial location. For example, the current arterial location may be imaged using one or more sensors of the BP monitoring device 500 (e.g., photoacoustic, ultrasonic, optical, camera, etc.). The current arterial location may correspond to some portion of an artery as determined based on a current orientation of the BP monitoring device 500. In general, the current arterial location may be imaged using any of the techniques described herein, such as those involving arterial tomographic mapping, internal microvascular structures, or dermatoglyphic skin patterns, to name some examples. According to some implementations, when imaging the current arterial location, one or more sensors of the BP monitoring device 500 may capture at least one image segment of the current arterial location. The image segment may correspond to a set of frames 546 of the current arterial location that were captured by sensors of the BP monitoring device 500 over some period of time (e.g., 5 seconds, 10 seconds, etc.).

In this example, block 550 involves determining location similarity scores. According to some implementations, for each of the arterial locations 512, 514, and 516, location similarity scores are determined for each of the set of frames 546 for the current arterial location with respect to corresponding calibration frames 548 that were determined for the arterial locations 512, 514, and 516 at block 526 of FIG. 5B. In this example, a first set of location similarity scores may be determined for each of the set of frames 546 for the current arterial location with respect to the calibration frame 548 that was determined for the arterial location 512, a second set of location similarity scores may be determined for each of the set of frames 546 for the current arterial location with respect to the calibration frame 548 that was determined for the arterial location 514, and a third set of location similarity scores may be determined for each of the set of frames 546 for the current arterial location with respect to the calibration frame 548 that was determined for the arterial location 516. In some implementations, a location similarity score may be determined based on any of the techniques described herein, such as a Structural Similarity Index Measure (SSIM) technique.

In this example, block 552 involves combining the location similarity scores. According to some implementations, the location similarity scores are averaged to produce a combined location similarity score. In this example, the first set of location similarity scores corresponding to the arterial location 512 may be combined into a first combined location similarity score, the second set of location similarity scores corresponding to the arterial location 514 may be combined into a second combined location similarity score, and the third set of location similarity scores corresponding to the arterial location 516 may be combined into a third combined location similarity score.

In this example, block 554 involves determining whether any of the combined location similarity scores for the arterial locations 512, 514, and 516 satisfy a threshold value. For example, an SSIM score may range from −1 to 1, where 1 indicates perfect similarity between a pair of frames. For example, the threshold value could be 0.5, 0.75, or 0.9. In this example, the first combined location similarity score for the arterial location 512 may be evaluated based on the threshold value. In this example, the second combined location similarity score for the arterial location 514 may be evaluated based on the threshold value. Further, in this example, the third combined location similarity score for the arterial location 516 may be evaluated based on the threshold value. In this example, if it is determined in block 554 that any one of the combined location similarity scores satisfies the threshold value, method 540 may proceed to block 556. Alternatively, if it is determined in block 554 that the combined location similarity score does not satisfy the threshold value, method 540 may proceed to block 558.

In this example, block 556 may involve confirming that a current orientation of the BP monitoring device 500 is aligned with an orientation of the BP monitoring device 500 at the time of calibration. In this example, if the first combined location similarity score satisfies the threshold value in block 554, a determination may be made that the current arterial location imaged by the BP monitoring device 500 at the time of initiating the BP test in block 542 corresponds to the arterial location 512 that was imaged by the BP monitoring device 500 at the time of BP calibration. Based on the correspondence, a determination can be made that the current orientation of the BP monitoring device 500 is aligned with the orientation of the BP monitoring device 500 when calibrated at the arterial location 512.

Similarly, in this example, if the second combined location similarity score satisfies the threshold value in block 554, a determination may be made that the current arterial location imaged by the BP monitoring device 500 at the time of initiating the BP test in block 542 corresponds to the arterial location 514 that was imaged by the BP monitoring device 500 at the time of BP calibration. Based on the correspondence, a determination can be made that the current orientation of the BP monitoring device 500 is aligned with the orientation of the BP monitoring device 500 when calibrated at the arterial location 514.

Further, in this example, if the third combined location similarity score satisfies the threshold value in block 554, a determination may be made that the current arterial location imaged by the BP monitoring device 500 at the time of initiating the BP test in block 542 corresponds to the arterial location 516 that was imaged by the BP monitoring device 500 at the time of BP calibration. Based on the correspondence, a determination can be made that the current orientation of the BP monitoring device 500 is aligned with the orientation of the BP monitoring device 500 when calibrated at the arterial location 516.

In some implementations, if more than one combined location similarity score satisfies the threshold value, then the calibrated arterial location 512, 514, or 516 that most closely aligns with the current arterial location may be used for BP estimation.

According to some implementations, upon confirming alignment, the BP monitoring device 500 may be configured to perform a blood pressure (BP) estimation. In some implementations, the BP estimation may be determined based on the ground truth BP associated with the aligned arterial location and a measurement of an arterial cross-sectional area associated with the current arterial location. For example, the BP estimation may be determined based, in part, by mapping a percentage change in the arterial cross-sectional area for the current arterial location to a percentage change in BP for the aligned arterial location.

In this example, block 558 may involve confirming that a current orientation of the BP monitoring device 500 is not aligned with an orientation of the BP monitoring device 500 at the time of calibration. According to some embodiments, user instructions for re-adjusting the BP monitoring device 500 may be provided. For example, the control system 301 may provide such instructions via the display system 302 associated with the BP monitoring device 500. In some implementations, the user instructions may include text-, voice-, or haptic-based live feedback that instructs the user to make changes to the orientation of the BP monitoring device 500. The instructions may ask the user to adjust the location or angle of the BP monitoring device 500. As another example, the instructions may ask the user to increase or decrease the tightness of a band associated with the BP monitoring device 500 to adjust counter pressure. As the user re-positions the BP monitoring device 500 to correspond to a different arterial location, method 540 may proceed again to block 544, which may involve imaging and evaluating the different arterial location for alignment, as described above. According to some implementations, user instructions asking the user to adjust the orientation of the BP monitoring device 500 may continue to be provided until a determination is made that the orientation of the BP monitoring device 500 is aligned with one of the calibrated arterial locations.

According to some embodiments, rather than asking the user to re-position the BP monitoring device 500, the control system 301 may compensate (or adjust) the current BP estimate based on a relationship between the current orientation of the BP monitoring device 500 and a calibrated arterial location that most closely aligns with the current orientation.

Although the approaches described herein concern alignment of device orientation for purposes of BP estimation, other applications are contemplated. For instance, according to some implementations, the approaches described herein may be applied for performing medical ultrasound, which can facilitate comparisons between scans using a handheld US imaging device while also providing an operator a reference to previous measurements. Moreover, the embodiments described herein may be implemented to be worn in different locations on the human body including, without limitation, a human wrist, digit, arm, or chest. Additionally, the embodiments described herein may be implemented to measure different arterial locations including, without limitation, a radial artery, an ulnar artery, a digital artery, or a brachial artery.

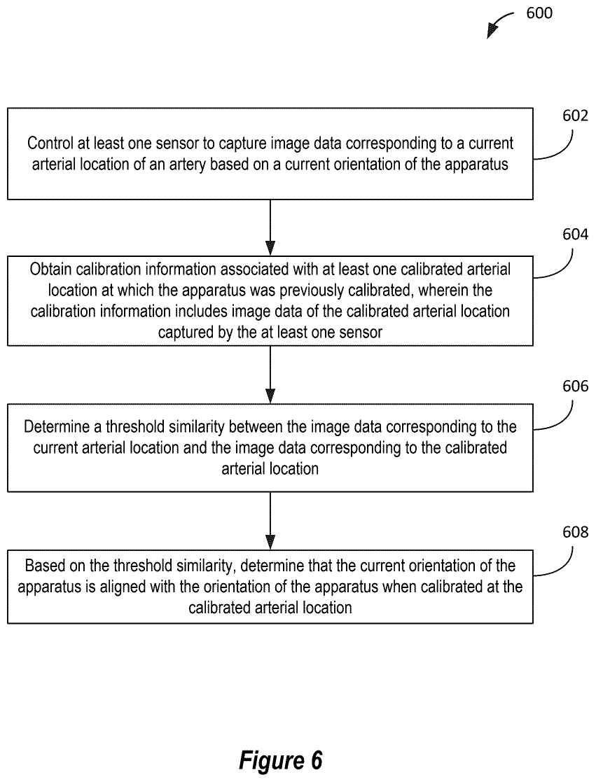

FIG. 6 is a flow diagram that provides example blocks of some methods disclosed herein. The blocks of FIG. 6 may, for example, be performed by the apparatus 300, or by a similar apparatus. As with other methods disclosed herein, the method 600 outlined in FIG. 6 may include more or fewer blocks than indicated. Moreover, the blocks of methods disclosed herein are not necessarily performed in the order indicated. In some examples, some blocks of methods disclosed herein may be performed concurrently or substantially concurrently.

In this example, block 602 involves controlling, by a control system of an apparatus, at least one sensor to capture image data corresponding to a current arterial location of an artery based on a current orientation of the apparatus.

According to this example, block 604 involves obtaining, by the control system, calibration information associated with at least one calibrated arterial location at which the apparatus was previously calibrated, wherein the calibration information includes image data of the calibrated arterial location captured by the at least one sensor.

In this example, block 606 involves determining, by the control system, a threshold similarity between the image data corresponding to the current arterial location and the image data corresponding to the calibrated arterial location.

In this example, block 608 involves determining, by the control system and based on the threshold similarity, that the current orientation of the apparatus is aligned with the orientation of the apparatus when calibrated at the calibrated arterial location.

According to some examples, the apparatus is configured to be worn on a human wrist and wherein the artery is a radial artery. In some examples, the control system is further configured to estimate a blood pressure for the current arterial location. In some examples, the blood pressure is estimated based at least in part on a measurement of arterial cross-sectional area at the current arterial location and a ground truth blood pressure measurement associated with the calibrated arterial location. In some examples, the at least one sensor is a photoacoustic sensor configured to capture image data representing arterial tomographic mapping. In some examples, the at least one sensor is an ultrasonic sensor configured to capture image data representing internal vascular structures. In some examples, the at least one sensor is an optical sensor configured to capture image data representing dermatoglyphic skin patterns. In some examples, the at least one sensor is a camera configured to capture image data representing dermatoglyphic skin patterns.

According to some examples, the control system is further configured to: detect a change from the current orientation of the apparatus to a new orientation, wherein, based on the change, the apparatus corresponds to a new arterial location; control the at least one sensor to capture image data corresponding to a new arterial location; determine that a threshold similarity between the image data corresponding to the new arterial location and the image data corresponding to the calibrated arterial location is not satisfied; and determine that the new orientation of the apparatus is not aligned with the orientation of the apparatus when calibrated at the calibrated arterial location. In some examples, the control system is further configured to provide user instructions to re-orient the apparatus via an interface. In some examples, the user instructions include instructions to adjust at least a position of the apparatus, an angle of the apparatus, or a counter pressure associated with the apparatus. In some examples, the apparatus is calibrated at a plurality of arterial locations including the at least one calibrated arterial location, and wherein the apparatus may be aligned with respect to any of the plurality of arterial locations.

Implementation examples are described in the following numbered clauses:

1. An apparatus, comprising: a control system configured to: control at least one sensor to capture image data corresponding to a current arterial location of an artery based on a current orientation of the apparatus; obtain calibration information associated with at least one calibrated arterial location at which the apparatus was previously calibrated, wherein the calibration information includes image data of the calibrated arterial location captured by the at least one sensor; determine a threshold similarity between the image data corresponding to the current arterial location and the image data corresponding to the calibrated arterial location; and based on the threshold similarity, determine that the current orientation of the apparatus is aligned with the orientation of the apparatus when calibrated at the calibrated arterial location.

2. The apparatus of clause 1, wherein the apparatus is configured to be worn on a human wrist, digit, arm, or chest.

3. The apparatus of clause 1 or clause 2, wherein the artery is a radial artery, an ulnar artery, a digital artery, or a brachial artery.

4. The apparatus of clauses 1-3, wherein the control system is further configured to estimate a blood pressure for the current arterial location.

5. The apparatus of clauses 1-4, wherein the blood pressure is estimated based at least in part on a measurement of arterial cross-sectional area at the current arterial location and a ground truth blood pressure measurement associated with the calibrated arterial location.

6. The apparatus of clauses 1-5, wherein the at least one sensor is a photoacoustic sensor configured to capture image data representing arterial tomographic mapping.

7. The apparatus of clauses 1-5, wherein the at least one sensor is an ultrasonic sensor configured to capture image data representing subdermal tissue structures and arterial cross-sectional area information.

8. The apparatus of clauses 1-5, wherein the at least one sensor is an optical sensor configured to capture image data representing dermatoglyphic skin patterns.

9. The apparatus of clauses 1-8, wherein the control system is further configured to: detect a change from the current orientation of the apparatus to a new orientation, wherein, based on the change, the apparatus corresponds to a new arterial location; control the at least one sensor to capture image data corresponding to a new arterial location; determine that a threshold similarity between the image data corresponding to the new arterial location and the image data corresponding to the calibrated arterial location is not satisfied; and determine that the new orientation of the apparatus is not aligned with the orientation of the apparatus when calibrated at the calibrated arterial location.

10. The apparatus of clauses 1-9, wherein the control system is further configured to provide user instructions to re-orient the apparatus via an interface.

11. The apparatus of clauses 1-10, wherein the user instructions include instructions to adjust at least a position of the apparatus, an angle of the apparatus, or a counter pressure associated with the apparatus.

12. The apparatus of clauses 1-11, wherein the apparatus is calibrated at a plurality of arterial locations including the at least one calibrated arterial location, and wherein the apparatus may be aligned with respect to any of the plurality of arterial locations.

13. A method, comprising: controlling, by a control system of an apparatus, at least one sensor to capture image data corresponding to a current arterial location of an artery based on a current orientation of the apparatus; obtaining, by the control system, calibration information associated with at least one calibrated arterial location at which the apparatus was previously calibrated, wherein the calibration information includes image data of the calibrated arterial location captured by the at least one sensor; determining, by the control system, a threshold similarity between the image data corresponding to the current arterial location and the image data corresponding to the calibrated arterial location; and based on the threshold similarity, determining, by the control system, that the current orientation of the apparatus is aligned with the orientation of the apparatus when calibrated at the calibrated arterial location.

14. The method of clause 13, wherein the at least one sensor is a photoacoustic sensor configured to capture image data representing arterial tomographic mapping.

15. The method of clause 13, wherein the at least one sensor is an ultrasonic sensor configured to capture image data representing internal vascular structures.

16. The method of clause 13, wherein the at least one sensor is an optical sensor configured to capture image data representing dermatoglyphic skin patterns.

17. One or more non-transitory computer-readable media having instructions for performing a method stored thereon, the method comprising: controlling, by a control system of an apparatus, at least one sensor to capture image data corresponding to a current arterial location of an artery based on a current orientation of the apparatus; obtaining, by the control system, calibration information associated with at least one calibrated arterial location at which the apparatus was previously calibrated, wherein the calibration information includes image data of the calibrated arterial location captured by the at least one sensor; determining, by the control system, a threshold similarity between the image data corresponding to the current arterial location and the image data corresponding to the calibrated arterial location; and based on the threshold similarity, determining, by the control system, that the current orientation of the apparatus is aligned with the orientation of the apparatus when calibrated at the calibrated arterial location.

18. The one or more non-transitory computer-readable media of clause 17, wherein the at least one sensor is a photoacoustic sensor configured to capture image data representing arterial tomographic mapping.

19. The one or more non-transitory computer-readable media of clause 17, wherein the at least one sensor is an ultrasonic sensor configured to capture image data representing internal vascular structures.

20. The one or more non-transitory computer-readable media of clause 17, wherein the at least one sensor is an optical sensor configured to capture image data representing dermatoglyphic skin patterns.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

1. An apparatus, comprising:
a control system configured to:
control at least one sensor to capture image data corresponding to a current arterial location of an artery based on a current orientation of the apparatus;
obtain calibration information associated with at least one calibrated arterial location at which the apparatus was previously calibrated, wherein the calibration information includes image data of the calibrated arterial location captured by the at least one sensor;
determine a threshold similarity between the image data corresponding to the current arterial location and the image data corresponding to the calibrated arterial location; and
based on the threshold similarity, determine that the current orientation of the apparatus is aligned with the orientation of the apparatus when calibrated at the calibrated arterial location.

2. The apparatus of claim 1, wherein the apparatus is configured to be worn on a human wrist, digit, arm, or chest.

3. The apparatus of claim 1, wherein the artery is a radial artery, an ulnar artery, a digital artery, or a brachial artery.

4. The apparatus of claim 1, wherein the control system is further configured to estimate a blood pressure for the current arterial location.

5. The apparatus of claim 4, wherein the blood pressure is estimated based at least in part on a measurement of arterial cross-sectional area at the current arterial location and a ground truth blood pressure measurement associated with the calibrated arterial location.

6. The apparatus of claim 1, wherein the at least one sensor is a photoacoustic sensor configured to capture image data representing arterial tomographic mapping.

7. The apparatus of claim 1, wherein the at least one sensor is an ultrasonic sensor configured to capture image data representing subdermal tissue structures and arterial cross-sectional area information.

8. The apparatus of claim 1, wherein the at least one sensor is an optical sensor configured to capture image data representing dermatoglyphic skin patterns.

9. The apparatus of claim 1, wherein the control system is further configured to:
detect a change from the current orientation of the apparatus to a new orientation, wherein, based on the change, the apparatus corresponds to a new arterial location;
control the at least one sensor to capture image data corresponding to a new arterial location;
determine that a threshold similarity between the image data corresponding to the new arterial location and the image data corresponding to the calibrated arterial location is not satisfied; and
determine that the new orientation of the apparatus is not aligned with the orientation of the apparatus when calibrated at the calibrated arterial location.

10. The apparatus of claim 9, wherein the control system is further configured to provide user instructions to re-orient the apparatus via an interface.

11. The apparatus of claim 10, wherein the user instructions include instructions to adjust at least a position of the apparatus, an angle of the apparatus, or a counter pressure associated with the apparatus.

12. The apparatus of claim 1, wherein the apparatus is calibrated at a plurality of arterial locations including the at least one calibrated arterial location, and wherein the apparatus may be aligned with respect to any of the plurality of arterial locations.

13. A method, comprising:
controlling, by a control system of an apparatus, at least one sensor to capture image data corresponding to a current arterial location of an artery based on a current orientation of the apparatus;
obtaining, by the control system, calibration information associated with at least one calibrated arterial location at which the apparatus was previously calibrated, wherein the calibration information includes image data of the calibrated arterial location captured by the at least one sensor;
determining, by the control system, a threshold similarity between the image data corresponding to the current arterial location and the image data corresponding to the calibrated arterial location; and
based on the threshold similarity, determining, by the control system, that the current orientation of the apparatus is aligned with the orientation of the apparatus when calibrated at the calibrated arterial location.

14. The method of claim 13, wherein the at least one sensor is a photoacoustic sensor configured to capture image data representing arterial tomographic mapping.

15. The method of claim 13, wherein the at least one sensor is an ultrasonic sensor configured to capture image data representing internal vascular structures.

16. The method of claim 13, wherein the at least one sensor is an optical sensor configured to capture image data representing dermatoglyphic skin patterns.

17. One or more non-transitory computer-readable media having instructions for performing a method stored thereon, the method comprising:
controlling, by a control system of an apparatus, at least one sensor to capture image data corresponding to a current arterial location of an artery based on a current orientation of the apparatus;
obtaining, by the control system, calibration information associated with at least one calibrated arterial location at which the apparatus was previously calibrated, wherein the calibration information includes image data of the calibrated arterial location captured by the at least one sensor;
determining, by the control system, a threshold similarity between the image data corresponding to the current arterial location and the image data corresponding to the calibrated arterial location; and
based on the threshold similarity, determining, by the control system, that the current orientation of the apparatus is aligned with the orientation of the apparatus when calibrated at the calibrated arterial location.

18. The one or more non-transitory computer-readable media of claim 17, wherein the at least one sensor is a photoacoustic sensor configured to capture image data representing arterial tomographic mapping.

19. The one or more non-transitory computer-readable media of claim 17, wherein the at least one sensor is an ultrasonic sensor configured to capture image data representing internal vascular structures.

20. The one or more non-transitory computer-readable media of claim 17, wherein the at least one sensor is an optical sensor configured to capture image data representing dermatoglyphic skin patterns.
